(19) United States
(12) Patent Application Publication
(10) Pub. No.: US 2026/0294384 A1
(43) Pub. Date: Oct. 1, 2026

(54) ULTRASOUND DIAGNOSTIC APPARATUS AND METHOD OF CONTROLLING ULTRASOUND DIAGNOSTIC APPARATUS

(71) Applicant: FUJIFILM Corporation, Tokyo (JP)

(72) Inventor: Kosei TAKEMOTO, Kanagawa (JP)

(73) Assignee: FUJIFILM Corporation, Tokyo (JP)

(21) Appl. No.: 19/565,537

(22) Filed: Mar. 13, 2026

(30) Foreign Application Priority Data
Mar. 31, 2025 (JP) 2025-058053

(51) Int. Cl. A61B 8/08; A61B 8/00

(52) U.S. Cl. CPC A61B 8/0883; A61B 8/461; A61B 8/467; A61B 8/5223

(57) ABSTRACT

Provided are an ultrasound diagnostic apparatus that can accurately capture an ultrasound image representing a parasternal left ventricular long axis view and a method of controlling an ultrasound diagnostic apparatus. An ultrasound diagnostic apparatus includes: an image recognition unit that recognizes a Valsalva sinus from an ultrasound image representing a parasternal left ventricular long axis view of a heart of a subject; a first evaluation unit that evaluates convexity of an anterior wall section of the Valsalva sinus recognized by the image recognition unit to acquire a first evaluation value; a second evaluation unit that evaluates linearity of a posterior wall section of the Valsalva sinus recognized by the image recognition unit to acquire a second evaluation value; and a notification unit that notifies a user of the first evaluation value acquired by the first evaluation unit and the second evaluation value acquired by the second evaluation unit.

### CROSS-REFERENCE TO RELATED APPLICATIONS

The present application claims priority under 35 U.S.C. § 119 to Japanese Patent Application No. 2025-058053, filed on Mar. 31, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a method of controlling an ultrasound diagnostic apparatus.

### 2. Description of the Related Art

In the related art, a so-called cardiac output is calculated by capturing an ultrasound image representing a tomographic plane of a heart of a subject by a so-called ultrasound diagnostic apparatus and analyzing the captured ultrasound image. The cardiac output is usually calculated by performing calculation steps of
(1) measuring a left ventricular outflow tract diameter in an ultrasound image representing a so-called parasternal left ventricular long axis view at a mid-systolic phase of the heart, and calculating a cross-sectional area of the left ventricular outflow tract,
(2) calculating a blood flow velocity-time integral of the left ventricular outflow tract using a so-called pulse Doppler method by using an ultrasound image representing a so-called apical five-chamber view or a so-called apical three-chamber view,
(3) calculating a so-called stroke volume by multiplying the cross-sectional area of the left ventricular outflow tract by the blood flow velocity-time integral of the left ventricular outflow tract, and
(4) calculating the cardiac output by multiplying the stroke volume by a heart rate. This technique is disclosed in, for example, JP1993-003869A (JP-H5-003869A).

### SUMMARY OF THE INVENTION

A user who is not skilled in capturing an ultrasound image representing a parasternal left ventricular long axis view may not be able to dispose an ultrasound probe at an appropriate position and an appropriate tilt angle in a case of capturing the ultrasound image. In this case, the left ventricular outflow tract diameter may not be accurately measured. Since a cross-sectional area of the left ventricular outflow tract is proportional to a square of the left ventricular outflow tract diameter, in a case where the left ventricular outflow tract diameter is not accurately measured, a calculation error of the cross-sectional area of the left ventricular outflow tract is large, and as a result, calculation errors of the stroke volume and the cardiac output may be large.

The present invention has been made to solve the above-described problem in the related art, and an object of the present invention is to provide an ultrasound diagnostic apparatus that can accurately capture an ultrasound image representing a parasternal left ventricular long axis view and a method of controlling an ultrasound diagnostic apparatus.

It is possible to achieve the object described above with the following configurations.
[1] An ultrasound diagnostic apparatus comprising: an image recognition unit that recognizes a Valsalva sinus from an ultrasound image representing a parasternal left ventricular long axis view of a heart of a subject; a first evaluation unit that evaluates convexity of an anterior wall section of the Valsalva sinus recognized by the image recognition unit to acquire a first evaluation value; a second evaluation unit that evaluates linearity of a posterior wall section of the Valsalva sinus recognized by the image recognition unit to acquire a second evaluation value; and a notification unit that notifies a user of the first evaluation value acquired by the first evaluation unit and the second evaluation value acquired by the second evaluation unit.
[2] The ultrasound diagnostic apparatus according to [1], in which the first evaluation unit acquires a curvature of the anterior wall section as the first evaluation value.
[3] The ultrasound diagnostic apparatus according to [1] or [2], in which the second evaluation unit acquires a value obtained by multiplying a curvature of the posterior wall section by -1 as the second evaluation value.
[4] The ultrasound diagnostic apparatus according to any one of [1] to [3], further comprising: a monitor that displays the ultrasound image; and a display controller that displays the anterior wall section and the posterior wall section on the monitor in different display forms in accordance with the first evaluation value acquired by the first evaluation unit and the second evaluation value acquired by the second evaluation unit.
[5] The ultrasound diagnostic apparatus according to any one of [1] to [4], further comprising: an image evaluation unit that evaluates the ultrasound image based on the first evaluation value acquired by the first evaluation unit and the second evaluation value acquired by the second evaluation unit.
[6] The ultrasound diagnostic apparatus according to [5], in which the image recognition unit recognizes an aortic valve annulus from the ultrasound image, and the image evaluation unit evaluates the ultrasound image by taking into account a distance between a straight line closest to a left ventricle among straight lines connecting the anterior wall section and the posterior wall section of the Valsalva sinus recognized by the image recognition unit and the aortic valve annulus, in addition to the first evaluation value and the second evaluation value.
[7] A method of controlling an ultrasound diagnostic apparatus, the method comprising: recognizing a Valsalva sinus from an ultrasound image representing a parasternal left ventricular long axis view of a heart of a subject; evaluating convexity of an anterior wall section of the recognized Valsalva sinus to acquire a first evaluation value; evaluating linearity of a posterior wall section of the recognized Valsalva sinus to acquire a second evaluation value; and notifying a user of the acquired first evaluation value and the acquired second evaluation value.

According to the aspects of the present invention, the ultrasound diagnostic apparatus comprises: the image recognition unit that recognizes the Valsalva sinus from the ultrasound image representing the parasternal left ventricular long axis view of the heart of the subject; the first evaluation unit that evaluates the convexity of the anterior wall section of the Valsalva sinus recognized by the image recognition unit to acquire the first evaluation value; the second evaluation unit that evaluates the linearity of the posterior wall section of the Valsalva sinus recognized by the image recognition unit to acquire the second evaluation value; and the notification unit that notifies the user of the first evaluation value acquired by the first evaluation unit and the second evaluation value acquired by the second evaluation unit, so that the ultrasound image representing the parasternal left ventricular long axis view can be accurately captured.

### BRIEF DESCRIPTION OF THE DRAWINGS

FIG. 1 is a block diagram illustrating a configuration of an ultrasound diagnostic apparatus according to Embodiment 1 of the present invention.

FIG. 2 is a block diagram illustrating an internal configuration of a transmission/reception circuit according to Embodiment 1 of the present invention.

FIG. 3 is a block diagram illustrating an internal configuration of an image generation unit according to Embodiment 1 of the present invention.

FIG. 4 is a diagram illustrating an example of an ultrasound image representing a parasternal left ventricular long axis view.

FIG. 5 is a diagram schematically illustrating an example of a cross section of a Valsalva sinus.

FIG. 6 is a diagram schematically illustrating a first example of a longitudinal section of a Valsalva sinus.

FIG. 7 is a diagram schematically illustrating a second example of the longitudinal section of the Valsalva sinus.

FIG. 8 is a flowchart illustrating an operation of the ultrasound diagnostic apparatus according to Embodiment 1 of the present invention.

FIG. 9 is a block diagram illustrating a configuration of an ultrasound diagnostic apparatus according to Embodiment 2 of the present invention.

### DESCRIPTION OF THE PREFERRED EMBODIMENTS

Hereinafter, the embodiments of the invention will be described with reference to the accompanying drawings.

The following configuration requirements are described based on a representative embodiment of the present invention, but the present invention is not limited to such embodiments.

In addition, in this specification, a numerical range represented by "to" means a range including numerical values described before and after "to", both ends inclusive, as a lower limit value and an upper limit value.

In this specification, the terms "same" and "identical" include an error range generally acceptable in the technical field.

### Embodiment 1

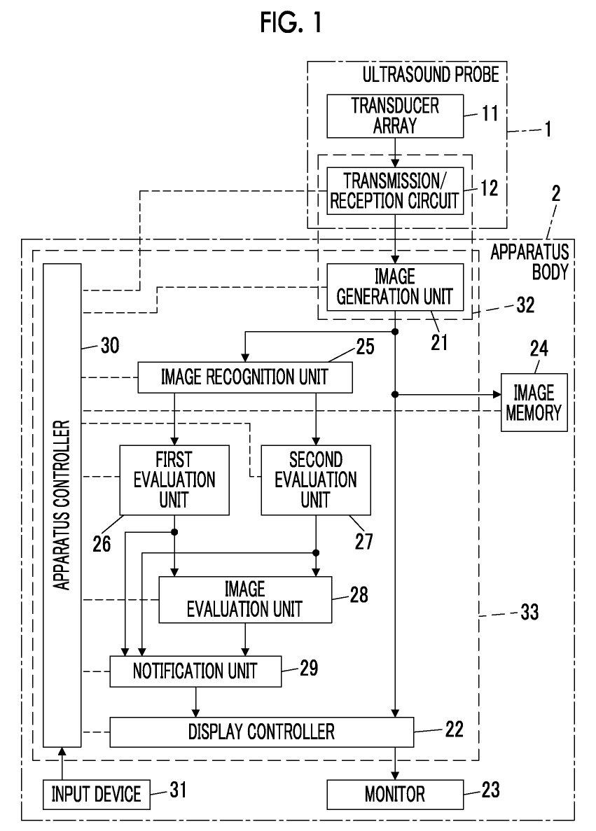

FIG. 1 illustrates a configuration of an ultrasound diagnostic apparatus according to Embodiment 1 of the present invention. The ultrasound diagnostic apparatus comprises an ultrasound probe 1 and an apparatus body 2 that are connected to each other through so-called wired communication or so-called wireless communication.

The ultrasound probe 1 comprises a transducer array 11, and a transmission/reception circuit 12 that is connected to the transducer array 11.

The apparatus body 2 comprises an image generation unit 21 that is connected to the transmission/reception circuit 12. In the apparatus body 2, a display controller 22 and a monitor 23 are sequentially connected to the image generation unit 21. An image memory 24 and an image recognition unit 25 are connected to the image generation unit 21. A first evaluation unit 26 and a second evaluation unit 27 are connected to the image recognition unit 25. An image evaluation unit 28 is connected to the first evaluation unit 26 and the second evaluation unit 27. A notification unit 29 is connected to the first evaluation unit 26, the second evaluation unit 27, and the image evaluation unit 28. The notification unit 29 is connected to the display controller 22. Further, an apparatus controller 30 is connected to the transmission/reception circuit 12, the image generation unit 21, the display controller 22, the image memory 24, the image recognition unit 25, the first evaluation unit 26, the second evaluation unit 27, the image evaluation unit 28, and the notification unit 29. An input device 31 is connected to the apparatus controller 30.

The transmission/reception circuit 12 and the image generation unit 21 form an image acquisition unit 32. Further, a processor 33 for the apparatus body 2 is configured by the image generation unit 21, the display controller 22, the image recognition unit 25, the first evaluation unit 26, the second evaluation unit 27, the image evaluation unit 28, the notification unit 29, and the apparatus controller 30.

The transducer array 11 of the ultrasound probe 1 has a plurality of ultrasound oscillators arranged in a one-dimensional or a two-dimensional manner. In accordance with a drive signal supplied from the transmission/reception circuit 12, each of the ultrasound oscillators transmits ultrasonic waves, receives an ultrasound echo from the subject, and outputs a signal based on the ultrasound echo. Each of the ultrasound oscillators is configured by forming, for example, electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

The image acquisition unit 32 including the transmission/reception circuit 12 and the image generation unit 21 acquires an ultrasound image in which the heart of the subject is imaged by transmitting and receiving the ultrasound beams using the ultrasound probe 1.

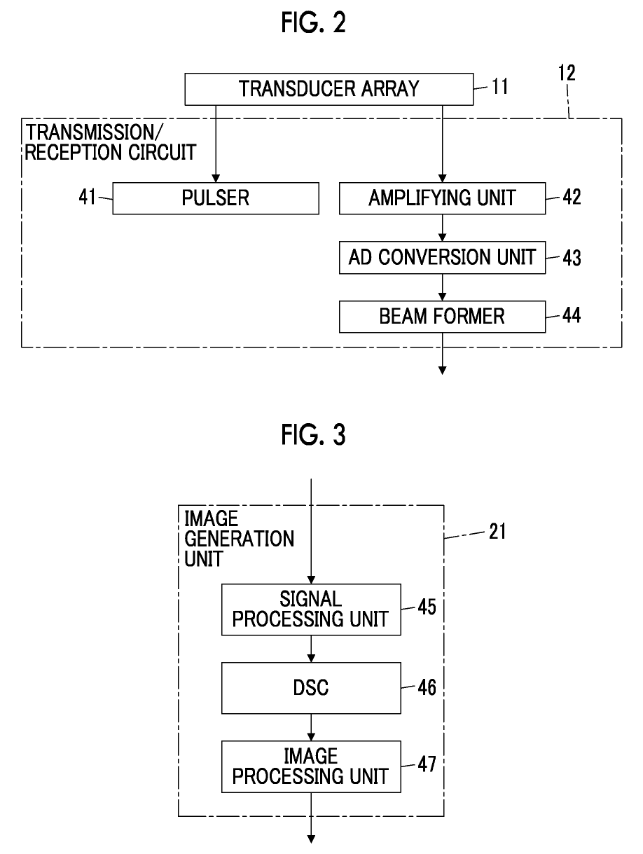

The transmission/reception circuit 12 transmits the ultrasonic waves from the transducer array 11 and generates a beamformed signal based on reception signals acquired by the transducer array 11 under control of the apparatus controller 30. As illustrated in FIG. 2, the transmission/reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, and the pulser 41 adjusts an amount of delay of each drive signal such that the ultrasound transmitted from the plurality of ultrasound oscillators of the transducer array 11 form the ultrasound beams, based on a transmission delay pattern selected in accordance with a control signal from the apparatus controller 30, and supplies the adjusted signal to the plurality of ultrasound oscillators. In a case where a pulsed or continuous wave-like voltage is applied to the electrodes of the ultrasound oscillator of the transducer array 11, the piezoelectric material expands and contracts to generate pulsed or continuous wave-like ultrasound from each of the ultrasound oscillators, whereby the ultrasound beams are formed from the combined wave of the ultrasound.

The transmitted ultrasound beam is reflected by the target such as a part of the subject, and propagates toward the transducer array 11 of the ultrasound probe 1. The ultrasound echo propagating toward the transducer array 11 as described above is received by each of the ultrasound oscillators constituting the transducer array 11. In this case, each of the ultrasound oscillators constituting the transducer array 11 receives the propagating ultrasound echo to expand and contract to generate the reception signal, which is an electrical signal, and outputs these reception signals to the amplifying unit 42.

The amplifying unit 42 amplifies the signal input from each of the ultrasound oscillators constituting the transducer array 11, and transmits the amplified signal to the AD conversion unit 43. The AD conversion unit 43 converts the signal transmitted from the amplifying unit 42 into digital reception data. The beam former 44 performs so-called receive focus processing by applying respective delays to the reception data received from the AD conversion unit 43, and then adding the delayed data together. By the receive focus processing, each reception data, which is converted by the AD conversion unit 43, is added in phase, and the beamformed signal in which the focus of the ultrasound echo is narrowed down is acquired.

As illustrated in FIG. 3, the image generation unit 21 has a configuration in which a signal processing unit 45, a digital scan converter (DSC) 46, and an image processing unit 47 are sequentially connected in series.

The signal processing unit 45 generates a B-mode image signal, which is tomographic image information related to tissues inside the subject, by performing, on the beamformed signal received from the transmission/reception circuit 12, correction of the attenuation due to a distance in accordance with a depth of a reflection position of the ultrasound by using a sound velocity value set by the apparatus controller 30 and then performing envelope detection processing.

The DSC 46 converts (raster-converts) the B-mode image signal generated by the signal processing unit 45 into the image signal in accordance with a standard television signal scanning method.

The image processing unit 47 performs various necessary image processing, such as gradation processing, on the B-mode image signal input from the DSC 46, and then transmits the B-mode image signal to the display controller 22, the image memory 24, and the image recognition unit 25. Hereinafter, the B-mode image signal, which has been image-processed by the image processing unit 47, will be referred to as an ultrasound image.

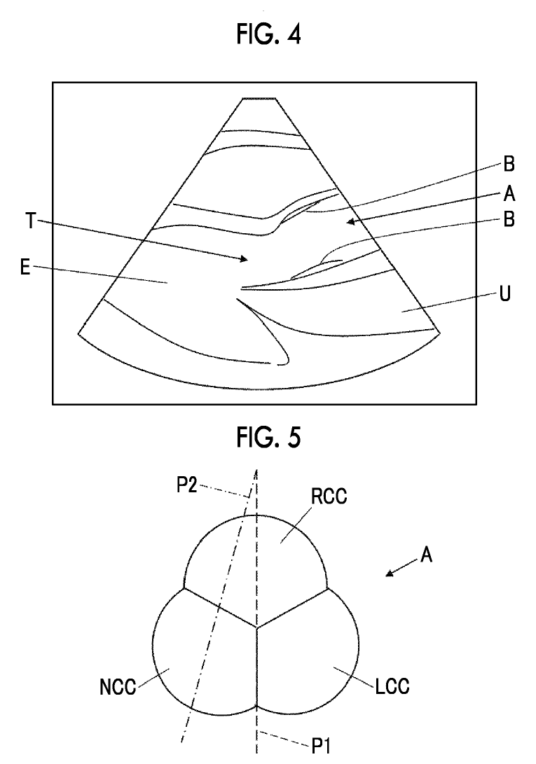

In the present invention, the image acquisition unit 32 acquires an ultrasound image U representing a parasternal left ventricular long axis view schematically illustrated in FIG. 4. The parasternal left ventricular long axis view is a cross section including a left ventricle E, a left ventricular outflow tract T, a left atrium, and a right ventricle. An aortic valve annulus is located in the left ventricular outflow tract T. The ultrasound image U representing the parasternal left ventricular long axis view is used, for example, to measure a left ventricular outflow tract diameter that is a diameter of the left ventricular outflow tract T.

The image memory 24 is a memory in which the plurality of frames of ultrasound images acquired by the image acquisition unit 32 are stored. The user can use, for example, the plurality of frames of ultrasound images stored in the image memory 24 to confirm the ultrasound images after an examination. As the image memory 24, for example, a recording medium such as a flash memory, a hard disk drive (HDD), a solid-state drive (SSD), a flexible disk (FD), a magneto-optical disk (MO disk), a magnetic tape (MT), a random-access memory (RAM), a compact disc (CD), a digital versatile disc (DVD), a secure digital card (SD card), or a universal serial bus memory (USB memory) can be used.

The image recognition unit 25 recognizes a so-called Valsalva sinus A from the ultrasound image U by analyzing the ultrasound image U representing the parasternal left ventricular long axis view of the heart of the subject. The Valsalva sinus A is a cavity section that is located at a position farther from the left ventricle E than the aortic valve annulus in the left ventricular outflow tract T. The image recognition unit 25 can recognize the Valsalva sinus A from the ultrasound image U by a so-called template matching method of searching for the ultrasound image U using a typical image pattern representing the Valsalva sinus A, a method of using a trained model in so-called machine learning of learning a relationship between a large number of ultrasound images U and the Valsalva sinus A shown in the ultrasound images U in advance, or the like.

FIG. 5 schematically illustrates a cross section of the Valsalva sinus A. The cross section of the Valsalva sinus A refers to a cut cross section of the Valsalva sinus A perpendicular to a running direction of the left ventricular outflow tract T. In general, it is known that the aortic valve has three types of valves, that is, a right coronary cusp RCC, a left coronary cusp LCC, and a non-coronary cusp NCC. In order to accurately measure the left ventricular outflow tract diameter in the ultrasound image U representing the parasternal left ventricular long axis view, it is ideal to capture the ultrasound image U corresponding to a cross section P1 that vertically cuts the right coronary cusp RCC through a boundary between the left coronary cusp LCC and the non-coronary cusp NCC. In the ultrasound image U corresponding to the cross section that does not pass through the boundary between the left coronary cusp LCC and the non-coronary cusp NCC as in a cross section P2 illustrated in FIG. 5, the left ventricular outflow tract diameter cannot be accurately measured.

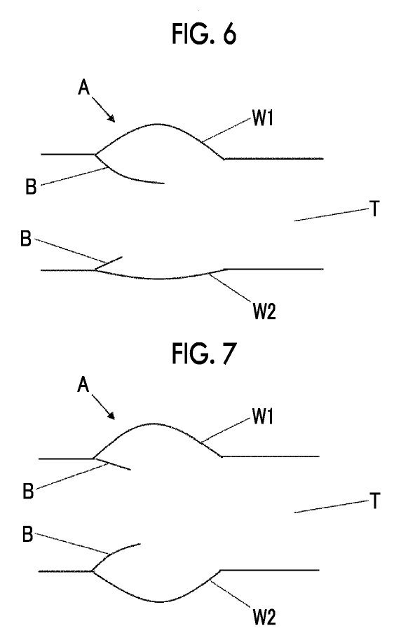

In the ultrasound image U corresponding to the cross section P1 that vertically cuts the right coronary cusp RCC through the boundary between the left coronary cusp LCC and the non-coronary cusp NCC, as illustrated in FIG. 6, an anterior wall section W1 of the Valsalva sinus A has a shape that is convex upward in a direction orthogonal to the running direction of the left ventricular outflow tract T, and a posterior wall section W2 of the Valsalva sinus A has a flat shape along the running direction of the left ventricular outflow tract T as compared with the anterior wall section W1. In the cross section P2 that does not pass through the boundary between the left coronary cusp LCC and the non-coronary cusp NCC, as illustrated in FIG. 7, the anterior wall section W1 of the Valsalva sinus A has a shape that is convex upward in a direction orthogonal to the running direction of the left ventricular outflow tract T, but the posterior wall section W2 of the Valsalva sinus A has a shape that is convex downward instead of a flat shape.

The first evaluation unit 26 evaluates the convexity of the anterior wall section W1 of the Valsalva sinus A recognized by the image recognition unit 25 to acquire a first evaluation value. The first evaluation unit 26 can calculate, for example, a curvature of the anterior wall section W1 in the ultrasound image U as the first evaluation value representing the convexity of the anterior wall section W1.

Here, the curvature of the anterior wall section W1 of the Valsalva sinus A to be calculated changes depending on a position and a tilt angle of the ultrasound probe 1. Here, the tilt angle of the ultrasound probe 1 means, for example, an angle formed by a body surface of the subject in contact with the ultrasound probe 1 and an orientation of the ultrasound probe 1, that is, a direction in which the ultrasound probe 1 is pressed against the body surface of the subject. The first evaluation unit 26 can also normalize the first evaluation value in a predetermined numerical range by, for example, setting a minimum value that can be taken by the first evaluation value based on the anatomical structure to 0, and setting a maximum value that can be taken by the first evaluation value based on the anatomical structure to 100.

The second evaluation unit 27 evaluates the linearity of the posterior wall section W2 of the Valsalva sinus A recognized by the image recognition unit 25 to acquire a second evaluation value. The second evaluation unit 27 can calculate, for example, a value obtained by multiplying the curvature of the posterior wall section W2 in the ultrasound image U by -1 as the second evaluation value representing the linearity of the posterior wall section W2.

The curvature of the posterior wall section W2 of the Valsalva sinus A to be calculated also changes depending on the position and the tilt angle of the ultrasound probe 1, as in the curvature of the anterior wall section W1. The second evaluation unit 27 can also normalize the second evaluation value in a predetermined numerical range by, for example, setting a minimum value that can be taken by the second evaluation value based on the anatomical structure to -100, and setting a maximum value that can be taken by the second evaluation value based on the anatomical structure to 0.

The image evaluation unit 28 evaluates the ultrasound image U based on the first evaluation value acquired by the first evaluation unit 26 and the second evaluation value acquired by the second evaluation unit 27. The image evaluation unit 28 can calculate an image evaluation value for the ultrasound image U by, for example, adding the normalized first evaluation value and the normalized second evaluation value and dividing the result by 2. A higher value of the image evaluation value represents that the cross section represented by the ultrasound image U is closer to the cross section P1 that passes through the boundary between the left coronary cusp LCC and the non-coronary cusp NCC, and a lower value of the image evaluation value represents that the cross section represented by the ultrasound image U is farther from the cross section P1 that passes through the boundary between the left coronary cusp LCC and the non-coronary cusp NCC. In addition, the image evaluation unit 28 has a plurality of evaluation threshold values for the image evaluation value, and can classify the image evaluation value into, for example, discrete evaluation results such as "good", "fair", and "bad" in a plurality of numerical ranges divided by the plurality of evaluation threshold values.

In addition, the image evaluation unit 28 can also cause a trained model in machine learning that has been trained using a relationship between the first evaluation value, the second evaluation value, and the image evaluation value to output the image evaluation value or the classification of the discrete evaluation results of the ultrasound image U by inputting the first evaluation value and the second evaluation value to the trained model.

The notification unit 29 notifies the user of the first evaluation value acquired by the first evaluation unit 26 and the second evaluation value acquired by the second evaluation unit 27. For example, the notification unit 29 can notify the user by displaying the first evaluation value and the second evaluation value on the monitor 23. Further, the notification unit 29 can also notify the user of the evaluation result of the ultrasound image U by the image evaluation unit 28, for example, by displaying the evaluation result on the monitor 23.

The user can easily capture the ultrasound image U representing the cross section P1 accurately by easily grasping whether or not the ultrasound image U currently being captured represents the cross section P1 that passes through the boundary between the left coronary cusp LCC and the non-coronary cusp NCC by confirming the content of the notification by the notification unit 29, and adjusting the position or the tilt angle of the ultrasound probe 1 in a case where the cross section represented by the ultrasound image U is farther away from the cross section P1.

The display controller 22 performs predetermined processing on the ultrasound image U acquired by the image acquisition unit 32 and the notification content by the notification unit 29, under the control of the apparatus controller 30, and then displays the processed image on the monitor 23. In addition, the display controller 22 can display the anterior wall section W1 and the posterior wall section W2 on the monitor 23 in different display forms in accordance with the first evaluation value acquired by the first evaluation unit 26 and the second evaluation value acquired by the second evaluation unit 27. The display controller 22 can display the anterior wall section W1 and the posterior wall section W2 such that the anterior wall section W1 and the posterior wall section W2 become closer to a first color such as red as the absolute value of the first evaluation value and the absolute value of the second evaluation value become larger, and become closer to a second color such as blue as the absolute value of the first evaluation value and the absolute value of the second evaluation value become smaller, for example. In addition, the display controller 22 can display the anterior wall section W1 and the posterior wall section W2 on the monitor 23 by changing a thickness, a transmittance, and the like of the contour of the anterior wall section W1 and the posterior wall section W2.

The monitor 23 displays the ultrasound image U acquired by the image acquisition unit 32 and the notification content by the notification unit 29 under the control of the display controller 22, and includes, for example, a display device such as a liquid-crystal display (LCD) or an organic electroluminescence display (organic EL display).

The input device 31 is an input device used by the user to perform an input operation, and is configured by, for example, devices such as a keyboard, a mouse, a trackball, a touchpad, and a touch sensor disposed in a state of being superimposed on the monitor 23.

In this embodiment, each processing in the processor 33 is executed by any computer. Any computer may execute these kinds of processing by the processor as hardware, a program as software, or a combination of the processor and the program. In this case, the processor is configured to execute various kinds of processing in this embodiment in cooperation with the program, and may function as each unit or each means in this embodiment. Further, the execution order of the processing by the processor is not limited to the above-described order and may be changed as appropriate. Any computer may mean a general-purpose computer, a computer for specific use, a workstation, or another system that can execute each processing.

The processor 33 may be configured by one or more hardware components, and the types of components are not limited. For example, the processor 33 may be configured by a programmable logic device such as a central processing unit (CPU), a microprocessing unit (MPU), or a field programmable gate array (FPGA), a dedicated circuit for executing specific processing, such as an application-specific integrated circuit (ASIC), or hardware such as a graphics processing unit (GPU) or a neural processing unit (NPU). Furthermore, the type of the hardware component may be a combination of different types of hardware components. In a case where the plurality of hardware components are configured to execute one or a plurality of kinds of processing of a certain processor, the plurality of hardware components may be present in devices physically separated from each other or may be present in the same device. Further, in any of the embodiments, the order of each processing performed by the processor 33 is not limited to the order described above, and may be changed as appropriate. In addition, the hardware is implemented in a form of an electrical circuit (circuitry) in which circuit elements, such as semiconductor elements, are combined.

Furthermore, the program may be software such as firmware or microcode. The program may be, for example, a group of program modules, and each function may be implemented by a processor configured to execute each function. The program may be a program code or a plurality of code segments stored in one or more non-transitory computer-readable media (for example, a storage media and other storages). The program may be stored in the plurality of non-transitory computer-readable media existing in devices physically separated from each other. The program code or the code segment may represent a procedure, function, subprogram, routine, subroutine, module, software package, class, or any combination of instructions, data structures, or program statements. The program code or the code segment may be connected to another code segment or a hardware circuit by the transmission and reception of information, data, arguments, parameters, or contents in the memory.

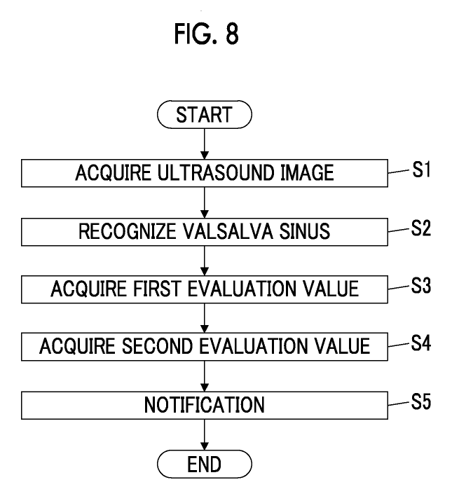

Hereinafter, an operation of the ultrasound diagnostic apparatus according to the embodiment will be described with reference to the flowchart illustrated in FIG. 8.

In step S1, the user disposes the ultrasound probe 1 on the body surface of the subject, and the image acquisition unit 32 acquires the ultrasound image U representing the parasternal left ventricular long axis view of the heart of the subject. In such a case, under the control of the apparatus controller 30, the transmission and reception of the ultrasound from the plurality of transducers of the transducer array 11 are started in accordance with the drive signal from the pulser 41 of the transmission/reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The receive focus processing is performed on the reception data by the beam former 44, the beamformed signal generated by the receive focus processing is transmitted to the image generation unit 21 of the apparatus body 2, and thus the ultrasound image U is generated by the image generation unit 21. In this case, the signal processing unit 45 of the image generation unit 21 performs the correction of the attenuation in accordance with the depth of the reflection position of the ultrasound and the envelope detection processing on the beamformed signal, the DSC 46 performs the conversion into the image signal in accordance with the normal television signal scanning method, and the image processing unit 47 performs various types of necessary image processing such as gradation processing.

In step S2, the image recognition unit 25 recognizes the so-called Valsalva sinus A from the ultrasound image U by analyzing the ultrasound image U acquired in step S1. The image recognition unit 25 can recognize the Valsalva sinus A from the ultrasound image U by, for example, the template matching method or the method of using the trained model in machine learning.

In step S3, the first evaluation unit 26 evaluates the convexity of the anterior wall section W1 of the Valsalva sinus A recognized in step S2 to acquire the first evaluation value. The first evaluation unit 26 can calculate, for example, the curvature of the anterior wall section W1 as the first evaluation value representing the convexity of the anterior wall section W1 of the Valsalva sinus A.

In step S4, the second evaluation unit 27 evaluates the linearity of the posterior wall section W2 of the Valsalva sinus A recognized in step S2 to acquire the second evaluation value. The second evaluation unit 27 can calculate, for example, the value obtained by multiplying the curvature of the posterior wall section W2 by -1 as the second evaluation value representing the linearity of the posterior wall section W2 of the Valsalva sinus A.

In step S5, the notification unit 29 notifies the user of the first evaluation value acquired in step S3 and the second evaluation value acquired in step S4. For example, the notification unit 29 can notify the user by displaying the first evaluation value and the second evaluation value on the monitor 23. The user can easily capture the ultrasound image U representing the cross section P1 accurately by easily grasping whether or not the ultrasound image U currently being captured represents the cross section P1 that passes through the boundary between the left coronary cusp LCC and the non-coronary cusp NCC by confirming the content of the notification by the notification unit 29, and adjusting the position or the tilt angle of the ultrasound probe 1 in a case where the cross section represented by the ultrasound image U is farther away from the cross section P1.

In a case where the processing of step S5 is completed in this way, the operation of the ultrasound diagnostic apparatus in accordance with the flowchart of FIG. 8 is completed.

As described above, with the ultrasound diagnostic apparatus of Embodiment 1 of the present invention, the image recognition unit 25 recognizes the Valsalva sinus A from the ultrasound image U representing the parasternal left ventricular long axis view of the heart of the subject, the first evaluation unit 26 evaluates the convexity of the anterior wall section W1 of the Valsalva sinus A recognized by the image recognition unit 25 to acquire the first evaluation value, the second evaluation unit 27 evaluates the linearity of the posterior wall section W2 of the Valsalva sinus A recognized by the image recognition unit 25 to acquire the second evaluation value, and the notification unit 29 notifies the user of the first evaluation value acquired by the first evaluation unit 26 and the second evaluation value acquired by the second evaluation unit 27, so that the user can accurately capture the ultrasound image U representing the parasternal left ventricular long axis view.

Further, the case has been described in which the transmission/reception circuit 12 is provided in the ultrasound probe 1, but the transmission/reception circuit 12 may be provided in the apparatus body 2.

Furthermore, the case has been described in which the image generation unit 21 is provided in the apparatus body 2, but the image generation unit 21 may be provided in the ultrasound probe 1.

The apparatus body 2 may be a so-called stationary type, a portable type that is easy to carry, or a so-called handheld type that is formed by, for example, a smartphone or a tablet type computer. In this way, the types of the devices forming the apparatus body 2 are not particularly limited.

In addition, in a case where the ultrasound diagnostic apparatus comprises a server (not illustrated) installed at a remote location from the apparatus body 2, the processor 33 can be provided in the server instead of being provided in the apparatus body 2.

The first evaluation unit 26 and the second evaluation unit 27 can also weight the first evaluation value and the second evaluation value. For example, since the shape of the posterior wall section W2 of the Valsalva sinus A changes more significantly than the shape of the anterior wall section W1 of the Valsalva sinus A in the ultrasound image U with respect to the change in the position and the tilt angle of the ultrasound probe 1, the first evaluation unit 26 and the second evaluation unit 27 can weight the first evaluation value and the second evaluation value such that a weight coefficient for the second evaluation value becomes larger than a weight coefficient for the first evaluation value. As the weight coefficient for each of the first evaluation value and the second evaluation value, for example, a predetermined value can be used.

### Embodiment 2

The image recognition unit 25 can recognize the aortic valve annulus in addition to the Valsalva sinus A. In this case, the image evaluation unit 28 can evaluate the ultrasound image U by taking into account the recognition of the aortic valve annulus by the image recognition unit 25.

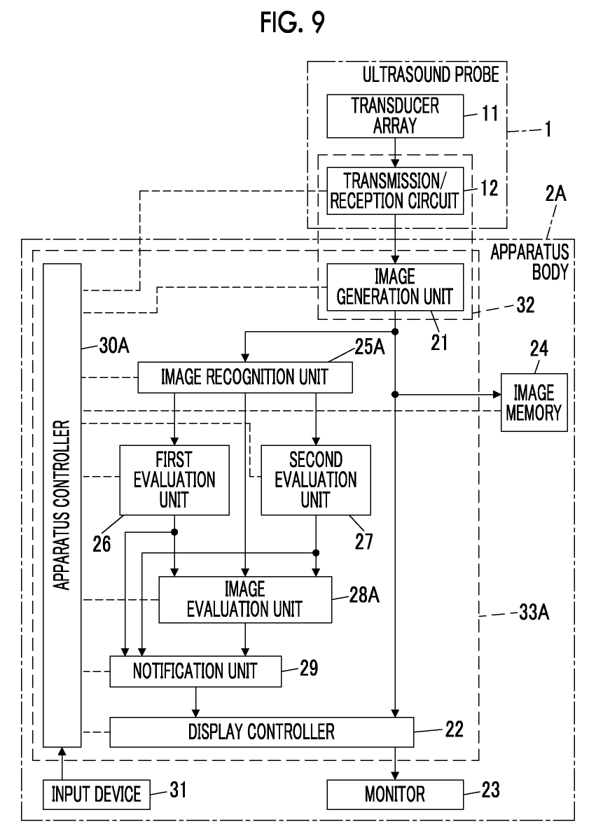

FIG. 9 illustrates a configuration of an ultrasound diagnostic apparatus according to Embodiment 2. The ultrasound diagnostic apparatus according to Embodiment 2 comprises an apparatus body 2A instead of the apparatus body 2, as compared with the ultrasound diagnostic apparatus according to Embodiment 1 illustrated in FIG. 1. The apparatus body 2A comprises an image recognition unit 25A instead of the image recognition unit 25, includes an image evaluation unit 28A instead of the image evaluation unit 28, and comprises an apparatus controller 30A instead of the apparatus controller 30, in the apparatus body 2 according to Embodiment 1.

The image recognition unit 25A is connected to the image generation unit 21 in the apparatus body 2A. The image evaluation unit 28A is connected to the image recognition unit 25A, the first evaluation unit 26, and the second evaluation unit 27. The image evaluation unit 28A is connected to the notification unit 29. The image recognition unit 25A and the image evaluation unit 28A are connected to the apparatus controller 30A. Further, a processor 33A for the apparatus body 2A is configured by the image generation unit 21, the display controller 22, the image recognition unit 25A, the first evaluation unit 26, the second evaluation unit 27, the image evaluation unit 28A, the notification unit 29, and the apparatus controller 30A.

The image recognition unit 25A recognizes the aortic valve annulus from the ultrasound image U, in addition to the Valsalva sinus A. The image recognition unit 25A can recognize the aortic valve annulus by, for example, the template matching method or the method of using the trained model in machine learning.

The image evaluation unit 28A evaluates the ultrasound image U by taking into account a distance between a straight line closest to the left ventricle E among straight lines connecting the anterior wall section W1 and the posterior wall section W2 of the Valsalva sinus A recognized by the image recognition unit 25A and the aortic valve annulus, in addition to the first evaluation value and the second evaluation value. The image evaluation unit 28A can calculate a final image evaluation value by, for example, adding or multiplying an evaluation correction value that becomes larger as the distance between the straight line closest to the left ventricle E among the straight lines connecting the anterior wall section W1 and the posterior wall section W2 and the aortic valve annulus becomes smaller to the image evaluation value calculated based on the first evaluation value and the second evaluation value. The image evaluation unit 28A can further classify the ultrasound image U into the discrete evaluations such as "good", "fair", and "bad" based on the calculated image evaluation value.

In addition, the image evaluation unit 28A can also cause a trained model in machine learning that has been trained to make the image evaluation value larger as the distance between the straight line closest to the left ventricle E among the straight lines connecting the anterior wall section W1 and the posterior wall section W2 and the aortic valve annulus is smaller to output the final image evaluation value in which the recognition result of the aortic valve annulus is taken into account by inputting the first evaluation value, the second evaluation value, and the ultrasound image U to the trained model. In this case, the image evaluation unit 28A can also cause the trained model to output the discrete evaluations such as "good", "fair", and "bad" for the ultrasound image U.

The image evaluation unit 28A can more accurately evaluate the ultrasound image U by taking into account the recognition result of the aortic valve annulus in this way than by evaluating the ultrasound image U based only on the first evaluation value and the second evaluation value.

As described above, with the ultrasound diagnostic apparatus of Embodiment 2, the image evaluation unit 28A evaluates the ultrasound image U by taking into account the distance between the straight line closest to the left ventricle E among the straight lines connecting the anterior wall section W1 and the posterior wall section W2 of the Valsalva sinus A recognized by the image recognition unit 25A and the aortic valve annulus, in addition to the first evaluation value and the second evaluation value, so that the ultrasound image U can be accurately evaluated. The user can accurately capture the ultrasound image U representing the parasternal left ventricular long axis view by confirming the evaluation result and adjusting the position or the tilt angle of the ultrasound probe 1.

### EXPLANATION OF REFERENCES

1: ultrasound probe

2, 2A: apparatus body

11: transducer array

12: transmission/reception circuit

21: image generation unit

22: display controller

23: monitor

24: image memory

25, 25A: image recognition unit

26: first evaluation unit

27: second evaluation unit

28, 28A: image evaluation unit

29: notification unit

30, 30A: apparatus controller

31: input device

32: image acquisition unit

33, 33A: processor

41: pulser

42: amplifying unit

43: AD conversion unit

44: beam former

45: signal processing unit

46: DSC

47: image processing unit

A: Valsalva sinus

B: aortic valve

E: left ventricle

LCC: left coronary cusp

NCC: non-coronary cusp

P1, P2: cross section

RCC: right coronary cusp

T: left ventricular outflow tract

U: ultrasound image

W1: anterior wall section

W2: posterior wall section

What is claimed is:

1. An ultrasound diagnostic apparatus comprising:
a processor configured to:
recognize a Valsalva sinus from an ultrasound image representing a parasternal left ventricular long axis view of a heart of a subject;
evaluate convexity of an anterior wall section of the Valsalva sinus to acquire a first evaluation value;
evaluate linearity of a posterior wall section of the Valsalva sinus to acquire a second evaluation value; and
notify a user of the first evaluation value and the second evaluation value.

2. The ultrasound diagnostic apparatus according to claim 1,
wherein the processor is configured to acquire a curvature of the anterior wall section as the first evaluation value.

3. The ultrasound diagnostic apparatus according to claim 1,
wherein the processor is configured to acquire a value obtained by multiplying a curvature of the posterior wall section by -1 as the second evaluation value.

4. The ultrasound diagnostic apparatus according to claim 2,
wherein the processor is configured to acquire a value obtained by multiplying a curvature of the posterior wall section by -1 as the second evaluation value.

5. The ultrasound diagnostic apparatus according to claim 1, further comprising:
a monitor,
wherein the processor is configured to display the anterior wall section and the posterior wall section on the monitor in different display forms in accordance with the first evaluation value and the second evaluation value.

6. The ultrasound diagnostic apparatus according to claim 2, further comprising:
a monitor,
wherein the processor is configured to display the anterior wall section and the posterior wall section on the monitor in different display forms in accordance with the first evaluation value and the second evaluation value.

7. The ultrasound diagnostic apparatus according to claim 3, further comprising:
a monitor,
wherein the processor is configured to display the anterior wall section and the posterior wall section on the monitor in different display forms in accordance with the first evaluation value and the second evaluation value.

8. The ultrasound diagnostic apparatus according to claim 1,
wherein the processor is configured to evaluate the ultrasound image based on the first evaluation value and the second evaluation value.

9. The ultrasound diagnostic apparatus according to claim 2,
wherein the processor is configured to evaluate the ultrasound image based on the first evaluation value and the second evaluation value.

10. The ultrasound diagnostic apparatus according to claim 3,
wherein the processor is configured to evaluate the ultrasound image based on the first evaluation value and the second evaluation value.

11. The ultrasound diagnostic apparatus according to claim 5,
wherein the processor is configured to evaluate the ultrasound image based on the first evaluation value and the second evaluation value.

12. The ultrasound diagnostic apparatus according to claim 8,
wherein the processor is configured to:
recognize an aortic valve annulus from the ultrasound image; and
evaluate the ultrasound image by taking into account a distance between a straight line closest to a left ventricle among straight lines connecting the anterior wall section and the posterior wall section of the Valsalva sinus and the aortic valve annulus, in addition to the first evaluation value and the second evaluation value.

13. A method of controlling an ultrasound diagnostic apparatus, the method comprising:
recognizing a Valsalva sinus from an ultrasound image representing a parasternal left ventricular long axis view of a heart of a subject;
evaluating convexity of an anterior wall section of the recognized Valsalva sinus to acquire a first evaluation value;
evaluating linearity of a posterior wall section of the recognized Valsalva sinus to acquire a second evaluation value; and
notifying a user of the acquired first evaluation value and the acquired second evaluation value.
